SEQUENCE LISTING (10 sequences)

SEQ ID NO: 1 - DNA, 24 bp, Artificial Sequence
  SYNTHESIZED
acggtgatgacgcgagctacacca

SEQ ID NO: 2 - DNA, 19 bp, Artificial Sequence
  SYNTHESIZED
ggcaacacaagtgaactgc

SEQ ID NO: 3 - DNA, 18 bp, Artificial Sequence
  SYNTHESIZED
gcagccacccgtaggtgt

SEQ ID NO: 4 - DNA, 20 bp, Artificial Sequence
  SYNTHESIZED
aaaattcccctccacaatta

SEQ ID NO: 5 - DNA, 30 bp, Artificial Sequence
  SYNTHESIZED
agcactgccactgctgcggtttccccaaca

SEQ ID NO: 6 - DNA, 20 bp, Artificial Sequence
  SYNTHESIZED
ggcctcacccagtctacatt

SEQ ID NO: 7 - DNA, 22 bp, Artificial Sequence
  SYNTHESIZED
tgctgattgttatctagtatgg

SEQ ID NO: 8 - DNA, 34 bp, Artificial Sequence
  SYNTHESIZED
catcaccatcttcataggctacttgacctatagt

SEQ ID NO: 9 - DNA, 25 bp, Artificial Sequence
  SYNTHESIZED
aggtgaacggaagtgcacacggacc

SEQ ID NO: 10 - DNA, 21 bp, Artificial Sequence
  SYNTHESIZED
aagccgtcacgtagtgcgcca

(19) United States
(12) Patent Application Publication
(10) Pub. No.: US 2013/0158243 A1
(43) Pub. Date: Jun. 20, 2013

(54) PROCESS FOR CONJUGATION OF NHS ESTERS WITH OLIGONUCLEOTIDES

(71) Applicant: Robert William Ball, Haverhill (GB)

(72) Inventor: Robert William Ball, Haverhill (GB)

(73) Assignee: Sigma-Aldrich Co. LLC, St. Louis, MO (US)

(21) Appl. No.: 13/719,901

(22) Filed: Dec. 19, 2012

Related U.S. Application Data
(60) Provisional application No. 61/577,340, filed on Dec. 19, 2011.

(51) Int. Cl. C07H 1/00

(52) U.S. Cl. CPC C07H 1/00
USPC 536/23.1

(57) ABSTRACT

The present invention provides processes for the conjugation of NHS esters to amino-modified oligonucleotides. The processes provide the amino-modified oligonucleotide on a solid support such that conjugation can be carried out under conditions that can accommodate a wide variety of NHS esters and oligonucleotides.

### CROSS-REFERENCE TO RELATED APPLICATIONS

This application claims priority to U.S. Provisional Application No. 61/577,340, filed Dec. 19, 2011, which is hereby incorporated by reference in its entirety.

### FIELD OF THE INVENTION

The present invention relates to the field of oligonucleotide chemistry. More specifically, the invention relates to improved processes for conjugating NHS esters with oligonulceotides. Oligonucleotides produced using the processes of the invention are useful as research reagents, diagnostic reagents, and in therapeutics.

### BACKGROUND OF THE INVENTION

The use of oligonucleotides as research, diagnostic, and therapeutic agents is growing rapidly. In many applications, the oligonucleotides are conjugated with another entity. For example, conjugation may attach a detector group to the oligonucleotide such that the detectable group can be used to determine the presence or absence of an oligonucleotide in a sample. Oligonucleotides can also be conjugated with other molecular entities to modulate their properties for other biological, therapeutic, or synthetic needs. Conjugation can also link two or more oligonucleotides.

N-Hydroxysuccinimide (NHS) esters are linker molecules which can be used for a variety of conjugation reactions. Reaction with an NHS ester facilitates the conjugation of the group attached to the ester moiety to a compound capable of reacting with the NHS ester, such as an amine-containing compound. However, the use of NHS esters in conjugating oligonucleotides has been limited by the conditions typically required for the stability of the oligonucleotides in solution. Current processes typically require that the oligonucleotides are contacted with the NHS esters in an aqueous buffer. However, many potentially useful NHS esters have limited solubility in aqueous solutions. Moreover, buffered aqueous solutions suitable for the oligonucleotides typically have a pH higher than what is desirable for the stability of many NHS esters.

Thus, there remains a need for processes of conjugating NHS esters to oligonucleotides such that a wide variety of NHS esters and oligonucleotides can be accommodated.

### SUMMARY OF THE INVENTION

The present invention relates to a process for conjugating NHS esters to supported amino-modified oligonucleotides.

In one aspect, the present invention provides a process for conjugating an N-hydroxysuccinimide ester with an amino-modified oligonucleotide, where the process comprises contacting the N-hydroxysuccinimide ester with the amino-modified oligonucleotide, wherein the amino-modified oligonucleotide is non-covalently bound to an ion exchange support.

In another aspect, the present invention provides a process for conjugating an N-hydroxysuccinimide ester with an amino-modified oligonucleotide, where the process comprises contacting the N-hydroxysuccinimide ester with the amino-modified oligonucleotide bound to a solid support, wherein the process is conducted in a non-aqueous environment.

In yet another aspect, the present invention provides a process for producing an oligonucleotide comprising an R¹ moiety, the process comprising the steps of (a) contacting an amino-modified oligonucleotide with an ion exchange support such that the amino-modified oligonucleotide binds to the support; (b) contacting the supported amino-modified oligonucleotide with an N-hydroxysuccinimide ester comprising R¹; and (c) eluting the oligonucleotide comprising R¹, wherein R¹ is chosen from hydrocarbyl and substituted hydrocarbyl.

Other features and iterations of the invention are described in more detail herein.

### DETAILED DESCRIPTION OF THE INVENTION

Briefly, therefore, the invention provides for the conjugation of NHS esters to supported amino-modified oligonucleotides. Generally, the processes described can be conducted under conditions such that the process can accommodate a wide variety of NHS esters and oligonucleotides.

### I. Conjugation Reaction

One aspect of the invention is an amino-modified oligonucleotide. The term “oligonucleotide” as used herein refers to a plurality of linked nucleotides which may be either single-stranded or double-stranded. Nucleotides generally comprise a sugar, base, and one or more phosphate groups. Nucleotide bases may comprise purine and pyrimidine bases, including, but not limited to, adenine, cytosine, caffeine, isoguanine, guanine, hypoxanthine, theobromine, thymine, uracil, xanthine, and modifications thereof, including, for example, nucleotides with a 2′O-4′C-methylene bridge in their sugar portion. Modifications include, but are not limited to, those that provide other chemical groups that incorporate additional charge, polarizability, hydrogen bonding, electrostatic interaction, or functionality to the individual nucleotides or their corresponding bases, sugars, phosphates, or to the oligonucleotides as a whole. Such modifications include, but are not limited to, modified bases, 2′-position sugar modifications, 5-position pyrimidine modifications, 8-position purine modifications, modifications at cytosine exocyclic amines, methylations, bases that can be part of unusual base-pairing combinations such as the isobases isocytidine and isoguanosine and the other modifications known to those skilled in the art. In addition, the modifications include modified backbones of the oligonucleotides, examples being phosphorothioate DNA, methylphosphonate DNA and other modifications known to those skilled in the art as reviewed by Micklefield (2001) Current Medicinal Chemistry 8:1157-1179. In some aspects, the oligonucleotide is 3′ modified or 5′ modified with groups known in the art.

The oligonucleotides may vary in the number of nucleotide bases in the oligonucleotide. For example, the oligonucleotides may include about 3 bases, about 5 bases, about 10 bases, about, 25 bases, about 50 bases, about 100 bases, about 120 bases, about 150 bases, about 200 bases, about 250 bases, about 300 bases, about 350 bases, about 400 bases, or a range between and including any two of these values. In some embodiments, the oligonucleotides may comprise about 3 bases to about 400 bases. In other embodiments, the oligonucleotides may comprise about 10 bases to about 200 bases. In still another embodiment, the oligonucleotides may comprise from about 5 to about 120 bases. In an exemplary embodiment, the oligonucleotides may comprise about 50 bases.

Oligonucleotides may also be characterized by their weight in Daltons (Da), and a sample of oligonucleotides may be characterized by their mass-average molecular weight, which gives the average mass of a sample of oligonucleotides. As will be appreciated by those of skill in the art, the weight of individual oligonucleotides may vary depending on factors such as the composition of the bases, sugars, and phosphates, as well as the number of linked nucleotides. In some aspects, the mass-average molecular weight of the individual oligonucleotides is about 500 Da, about 1,500 Da, about 2,000 Da, about 5,000 Da, about 10,000 Da, about 20,000 Da, about 30,000 Da, about 40,000 Da, about 50,000 Da, about 100,000 Da, about 150,000 Da, about 200,000 Da, or a range between and including any two of these values. In various embodiments, the oligonucleotides have a mass-average molecular weight ranging from about 500 to about 200,000 Da. In one embodiment, the oligonucleotides have a mass-average molecular weight ranging from about 2,000 to about 20,000 Da. In an exemplary embodiment, the oligonucleotides have a mass-average molecular weight ranging from about 1,500 to about 40,000 Da.

The oligonucleotides may be amino-modified. By “amino-modified” it is meant that the oligonucleotide is modified with an amino group, or multiple amino groups, capable of reacting with an NHS ester. The modification may comprise the addition of a variety of moieties that comprise one or more primary or secondary amines, or the modification may comprise the addition of one or more primary or secondary amines. Thus, the amino-modification may comprise the general formula [O]-Zₙ, where [O] is the oligonucleotide; Z, at each occurrence, is chosen from NH₂, NHR, RNH₂, or RNHR; n is a non-zero integer; and R is independently and at each occurrence chosen from hydrocarbyl or substituted hydrocarbyl groups. For clarity, it is understood that where R is a divalent group, such a divalent R group may be a hydrocarbylene or substituted hydrocarbylene group including but not limited to an alkylene or substituted alkylene group. Where R is a monovalent group, R may be any number of hydrocarbyl or substituted hydrocarbyl groups, including but not limited to alkyl and substituted alkyl groups. R, when a monovalent group, may also be any number of protecting groups commonly known in the art, such as those described in Protective Groups in Organic Synthesis (Green, Wuts; 3^{rd} ed., 1999, John Wiley & Sons, Inc.), which is incorporated by reference for all purposes as if fully set forth herein. The oligonucleotide may be amino-modified at any position within the oligonucleotide. In some embodiments, the amino-modification is at the 5′ terminus. In other embodiments, the amino-modification is at the 3′ terminus. Oligonucleotides including amino-modified termini may be prepared using methods and reagents commonly employed in the art. For example, an oligonucleotide with an amino-modification at the 5′ terminus may be prepared using various linkers (also known in the art as modifiers) including but not limited to C2-C12 amino linkers or amino protected derivatives thereof. Examples of C2-C12 amino linkers or amino protected derivatives thereof include, but are not limited to, 2-[2-(4-monomethoxytrityl)aminoethoxy]ethyl(2-cyanoethyl)-N,N-diisopropyl)phosphoramidite (a C5 MMT amino linker), 6-(4-monomethoxytritylamino)hexyl[(2-cyanoethyl)-(N,N-diisopropyl)]phosphoramidite (a C6 MMT amino linker), 6-(trifluoroacetylamino)hexyl[(2-cyanoethyl)-(N,N-diisopropyl)]phosphoramidite (a C6 TFA amino linker), 7-(4-monomethoxytritylamino)heptyl[(2-cyanoethyl)-(N,N-diisopropyl)]phosphoramidite (a C7 MMT amino linker),12-(4-monomethoxytritylamino)dodecyl[(2-cyanoethyl)-(N,N-diisopropyl)]phosphoramidite (a C12 MMT amino linker). In yet other embodiments, the oligonucleotide includes one or more amino-modifications at a position other than the 3′ or the 5′ termini. For example, the oligonucleotide may include amino modifying groups directly attached, or otherwise tethered to one or more bases of the oligonucleotide. Such amino-modified oligonucleotides may be prepared using any number of phosphoramidites commonly known in the art, including but not limited to: an amino C6 dT (e.g., 5′-dimethoxytrityl-5-[N-(trifluoroacetylaminohexyl)-3-acrylimido]-2′-deoxyuridine, 3′-[(2-cyanoethyl)-(N,N-diisopropyl)]phosphoramidite, 5′-dimethoxytrityl-5-[N-((9-fluorenylmethoxycarbonyl)-aminohexyl)-3-acrylimido]-2′-deoxyuridine,3′-[(2-cyanoethyl)-(N,N-diisopropyl)]phosphoramidite and the like); an amino C6 dA (e.g., 5′-dimethoxytrityl-N6-benzoyl-N8-[6-(trifluoroacetylamino)-hex-1-yl]-8-amino-2′-deoxyadenosine-3′-[(2-cyanoethyl)-(N,N-diisopropyl)]phosphoramidite); an amino C6 dG (e.g., 5′-dimethoxytrityl-N2-(N,N-dimethylaminomethylidene)-N8-[6-(trifluoroacetylamino)-hex-1-yl]-8-amino-2′-deoxyguanosine-3′-[(2-cyanoethyl)-(N,N-diisopropyl)]phosphoramidite), an amino C6 dC (e.g., 5′-dimethoxytrityl-N-dimethylformamidine-5-[N-(trifluoroacetylaminohexyl)-3-acrylimido]-2′-deoxycytidine,3′-[(2-cyanoethyl)-(N,N-diisopropyl)]phosphoramidite), and/or an amino C2 dT (e.g., 5′-dimethoxytrityl-5-[N-(trifluoroacetylaminoethyl)-3-acrylimido]-2′-deoxyuridine,3′-[(2-cyanoethyl)-(N,N-diisopropyl)]phosphoramidite). For clarity, it is understood that any phosphoramidites which include protected amino groups, including but not limited to those indicated above, may be optionally deprotected after (or during) the synthesis of the amino-modified oligonucleotide, using deprotection methods known in the art.

The oligonucleotides of the invention may further be bound, preferably releasably, to a solid support. The term “solid support” as used herein refers to a resin, or solid support material, that is insoluble in the media employed in the reaction. In various aspects, the oligonucleotides may be bound to the solid support material through covalent or non-covalent interactions. Covalent bonding is characterized by shared electrons. In some embodiments the oligonucleotides are bound to the ion exchange resin by non-covalent bonding such as, for example dipole-dipole interactions, hydrophobic interactions, ionic interactions, London dispersion forces, Van der Waals forces, hydrogen bonding, and the like.

The solid support material may be of any type known in the art including those of various mesh and pore sizes. Solid support materials may comprise an inorganic polymer, including, but not limited to silica, alumina and controlled pore glass (CPG), or an organic polymer, including, but not limited to polystyrene, polyacrylamide, polymethacrylate, polyvinylalcohol, or other synthetic polymers, carbohydrates such as cellulose and starch or other polymeric carbohydrates, or other organic polymers and any copolymers, composite materials or combination of any of the above inorganic or organic materials. The solid support materials can be free in solution or be provided in a cartridge, column, or well, either as bound to the surface or packed within the column, cartridge, or well.

The solid support materials typically interact with the oligonucleotides through interactions on the surface of the solid support material. In some embodiments, the resins comprise linker molecules which bind to the oligonucleotides. In some embodiments, the solid support may be an ion exchange solid support, which is characterized by an ability to trap and release ions. The ion exchange support surface may comprise a strong base, a weak base, a strong acid or a weak acid exchanger. In preferred embodiments, the ion exchange support comprises a strong base exchanger surface moiety. Suitable strong base surface moieties may be selected from quaternary amine (i.e., ammonium) surface moieties, for example trimethylammonium ethyl (TMAE), or a tertiary amine, such as for example diethylaminoethane (DEAE). In a preferred embodiment, the solid support comprises an ion exchange resin and the oligonucleotides bind to the solid support through non-covalent interactions.

The process further comprises an NHS ester. Generally, NHS esters are of Formula (I) wherein R¹ is chosen from hydrocarbyl and substituted hydrocarbyl.

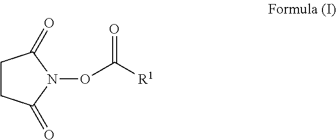

In some embodiments, R¹ may be a fluorescent moiety, a dye, or a label. These are any chemical moiety capable of imparting biological properties or detectability by spectroscopic, photochemical, biochemical, immunochemical, electrical, optical or chemical means. These moieties include, but are not limited to, biotin and biotin derivatives; chemiluminescent agents (such as, for example, acridinium esters, stabilized dioxetanes, and the like); fluorescein and its derivatives; rhodamine and its derivatives (such as, for example, carboxytetramethylrhodamine, tetramethylrhodamine and their derivatives); coumarin derivatives (such as, for example, methoxycoumarin, dialkylaminocoumarin, hydroxycoumarin and aminomethylcoumarin); BODIPY dyes; lanthanides; metal nanoparticles (such as, for example, gold, silver, copper and platinum); colorimetric labels (dyes, colloidal gold, and the like); magnetic labels (such as, for example magnetic nanoparticules) polymeric labels, and isotopically labeled compounds. In other embodiments, R¹ may be a biological macromolecule including oligonucleotides, glycoproteins, polysaccharides, proteins, peptides, immunogenic compounds, lipids, hormones, and the like. In some aspects of the invention, R¹ is chosen from 6-carboxy-x-rhodamine (ROX); tetramethylrhodamine (TAM); Alexa Fluor® 488 (A488); Alexa Fluor® 647 (A647); Texas Red ® (TxRd); Cy5 (Cy5); digoxigenin (DIG); LightCycler® 610 (LC610); LightCycler® 640 (LC640); and the like. In some aspects the compounds comprising Formula (I) may be purchased from commercial suppliers such as Roche, Invitrogen, Glen Research, and GE Healthcare.

Contacting the NHS ester with the solid supported amino-modified oligonucleotide causes a conjugation reaction between the amino group of the amino modified oligonucleotide and the NHS ester such that the R¹ group may be conjugated to the oligonucleotide by an amide bond as generally shown in Reaction Scheme 1.

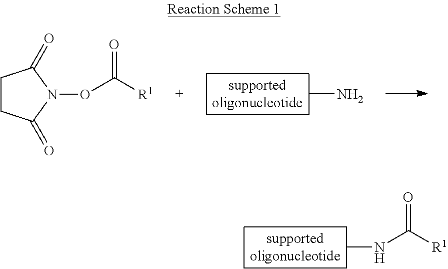

In some embodiments, the process comprises a solvent or a combination of solvents. In a preferred embodiment, the solvent is an organic solvent. Non-limiting examples of organic solvents are acetonitrile, acetone, allyl alcohol, benzene, butyl acetate, chlorobenzene, chloroform, chloromethane, cyclohexane, cyclopentane, dichloroethane, diethyl ether, dimethyl sulfoxide (DMSO), dimethylformamide, dioxane, ethanol, ethyl acetate, ethylene dichloride, ethylene bromide, formic acid, fluorobenzene, heptane, hexane, isobutylmethylketone, isopropanol, isopropyl acetate, N-methylpyrrolidone, methanol, methylene bromide, methylene chloride, methyl iodide, methyl ethyl ketone, 2-methyltetrahydrofuran, pentyl acetate, propanol, n-propyl acetate, sulfolane, tetrahydrofuran, tetrachloroethane, toluene, trichloroethane, xylene and combinations of any two or more thereof. In a preferred embodiment, the solvent is DMSO.

In some embodiments, the NHS ester is provided to the amino-modified supported oligonucleotide dissolved in a solvent. When dissolved in a solvent, the concentration of the NHS ester in the solvent may be provided in any range. For example, the concentration of the NHS ester in the solvent may be about 0.001 mg/μL, about 0.005 mg/μL, about 0.01 mg/μL, about 0.05 mg/μL, about 0.1 mg/μL, about 0.5 mg/μL, about 1 mg/μL, or a range between and including any two of these values. In some embodiments, the concentration of the NHS ester in a solvent may range from about 0.001 mg/μL to about 1 mg/μL. In other embodiments, the concentration of the NHS ester in a solvent may be about 0.001 mg/μL, about 0.005 mg/μL, about 0.01 mg/μL, about 0.05 mg/μL, about 0.1 mg/μL, about 0.5 mg/μL, or about 1 mg/μL. In an exemplary embodiment, the concentration of the NHS ester in a solvent is about 0.005 mg/μL or about 0.01 mg/μL.

In some embodiments, the conjugation reaction may be carried out in a non-aqueous environment. By non-aqueous environment, it is meant a liquid environment comprising less than about 5% water, less than about 4% water, less than about 3% water, less than about 2% water, or less than about 1% water. In some embodiments, water comprises less than about 0.5% of the liquid environment, or less than about 0.25% of the liquid environment, or less than about 0.1% of the liquid environment. In a preferred embodiment, water is present in an amount less than about 0.5%.

The temperature at which the conjugation is conducted may vary in different embodiments and over the course of the reaction. In some aspects, the conjugation reaction is conducted at a temperature of 0° C., 5° C., 10° C., 15° C., 20° C., 25° C., 30° C., 35° C., 40° C., 50° C., 60° C., 70° C., 80° C., or at a range between and including any two of these values. In one embodiment, the reaction is carried out at a temperature ranging between about 5° C. and about 50° C. In another embodiment, the temperature may range between about 10° C. and about 30° C. In an exemplary embodiment, conjugation is conducted at about 25° C.

The amount of time in which the conjugation reaction occurs may vary depending on the reactivity of the NHS ester, the reactivity of the amino-modified oligonucleotide, the nature of the support, concentration, temperature, as well as other factors. Generally, the reaction occurs over a period of time ranging from about 10 minutes to about 5 hours. In one embodiment, the reaction occurs over a period of time ranging from about 10 minutes to about 40 minutes. In still another embodiment, the reaction occurs over a period of time ranging from about 20 minutes to about 1 hour. In yet another embodiment, conjugation occurs over about 45 minutes.

In some embodiments, the conditions during the conjugation reaction are such that the amino moiety of the amino modified oligonucleotide is “free” i.e. the moiety is not protonated under the reaction conditions. Without being bound to any theory, this is thought that the conjugation reaction between the NHS ester and the amino-modified oligonucleotide is accelerated when the amino moiety of the amino modified oligonucleotide are free.

Generally, the conjugation reactions are performed at a pH of 8 or lower. In some embodiments, the pH may range from about 6 to about 8. In other embodiments, the pH may range from about 6 to about 6.5, from about 6.5 to about 7, or from about 7.5 to about 8. In one embodiment, the pH is about 7. In some aspects, the conjugation reaction is performed under neutral conditions.

### II. Process for Producing an Oligonucleotide Comprising R¹

In another aspect of the invention, a process for producing an R¹ derivatized oligonucleotide may be provided by (a) contacting an amino-modified oligonucleotide with an ion exchange support such that the amino-modified oligonucleotide binds to the support; (b) contacting the supported amino-modified oligonucleotide with an N-hydroxysuccinimide ester comprising R¹; and (c) eluting the oligonucleotide comprising R¹. The process provides an oligonucleotide comprising R¹, wherein R¹ is conjugated to the oligonucleotide through an amide bond. The process is generally illustrated by Reaction Scheme 2.

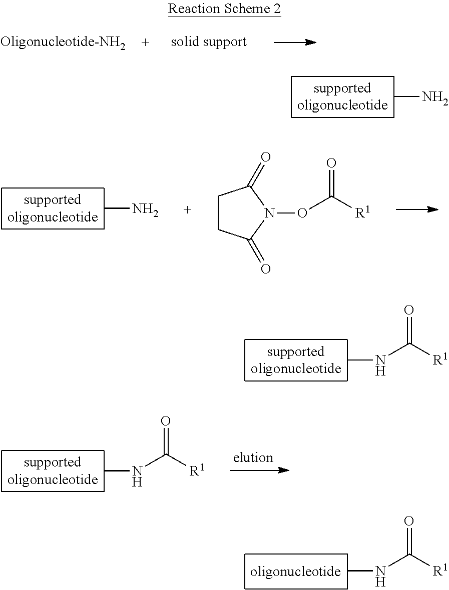

(a) Contacting an Amino-Modified Oligonucleotide with a Solid Support

The process involves contacting the amino-modified oligonucleotide with the solid support. Typically, the amino-modified oligonucleotides are dissolved in a solvent and provided to the solid support. The solvent may comprise any solvent without limitation including those listed in section (I). Additionally, the amino-modified oligonucleotide may be provided in an aqueous solution or a buffered aqueous solution. Suitable buffers, without limitation may include amine buffers, bicarbonate buffers, borate buffers, and carbonate buffers. Buffers may be provided with any acceptable counterion including, but not limited to lithium, sodium, potassium, calcium, magnesium, and the like.

In some aspects, the solid support materials are provided as beads and combined with the amino-modified oligonucleotides which are stirred or incubated with the amino-modified oligonucleotides for a sufficient time to allow binding through either covalent or non-covalent interactions. In other embodiments, the solid support is bound or packed in a cartridge, column or well. In an exemplary embodiment, the solid support material is packed in a column.

In some aspects, the solid-supported amino-modified oligonucleotides are washed, as is common in solid support syntheses. Washing is conducted with a solvent which does not significantly elute the oligonucleotide from the solid support. Washing steps may remove unbound oligonucleotides or may displace unwanted solvents. In a preferred embodiment, the solid-support is washed with an organic solvent to displace any water present with a non-aqueous solvent. Suitable solvents include, but are not limited to, those listed in section (I).

(b) Contacting the Supported Oligonucleotide with an N-hydroxysuccinimide Ester

The NHS ester may then be contacted with the solid supported amino-modified oligonucleotide. The NHS ester may be provided to the oligonucleotide as described in section (I).

### (c) Eluting the Oligonucleotide Comprising R¹

After the NHS ester and the supported amino-modified oligonucleotide are contacted for a sufficient period of time, the oligonucleotide comprising R¹ may be eluted. In some aspects of the invention, byproducts may be washed as described above from the solid support by prior to eluting the oligonucleotide comprising R¹. Elution of the oligonucleotide comprises contacting the bound oligonucleotide with an agent capable of disrupting or breaking the binding of the oligonucleotide to the support. This gives an unbound oligonucleotide comprising R¹, where R¹ is bound to the oligonucleotide through an amide bond.

Acceptable solvents for eluting the oligonucleotide can and will vary depending on the solid support and the type of linkage between the solid support and the oligonucleotide. In embodiments where the solid support is an ion exchange resin, the oligonucleotide may be eluted with a salt solution. The salt solution generally comprises a cation or anion capable of replacing the oligonucleotide. Acceptable cations include calcium, magnesium, sodium, potassium, silver, and ammonium. The counterion may be any known in the art including, but not limited to, acetate, fluoride, chloride, bromide, iodide, hydroxide, carbonate and bicarbonate.

The salt solution may be present in an aqueous or an organic solvent. Organic solvents may be selected from those know in the art, including, but not limited to solvents listed in section (I). When two or more solvents are present the solvents can be in any combination without limitation. The amount of salt present in the solution may also vary. In some aspects, the concentration of the salt solution may range between about 0.1M and about 10M. In other aspects, the concentration may range between about 1M and about 3 M. In a preferred embodiment, the salt solution is 2M solution of sodium chloride in a mixture of acetonitrile and water.

The eluted oligonucleotide comprising R¹ may be used as eluted or optionally purified by any means known in the art. In various aspects, the oligonucleotide may be purified by High Performance Liquid Chromatography (HPLC), ion-exchange chromatography, other types of chromatography, precipitation, crystallization, or polyacrylamide gel electrophoresis (PAGE) gels.

The yield of the oligonucleotide comprising R¹ can and will vary. In some embodiments, the yield may be at least about 50%. In one embodiment, the yield of the oligonucleotide comprising R¹ may range between about 65% and about 75%. In another embodiment, the yield of the conjugated oligonucleotide may range between about 75% and about 85%. In still another embodiment, the yield of the oligonucleotide comprising R¹ may be greater than about 85%. In another embodiment, the yield of the oligonucleotide comprising R¹ may range between about 85% and about 95%. In still another embodiment, the yield of the oligonucleotide comprising R¹ may be greater than about 95%.

### Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The terms “amine” and “amino” as used herein may refer to primary, secondary, or tertiary amines of the general formula R—NH₂, R,R′—NH, R,R¹—NR″ respectively, wherein R and R′ represent alkyl groups with 1 to 20 carbon atoms. The alkyl groups may be straight chain or branched chain alkyl groups or cycloalkyl groups with or without heteroatoms in the cycloalkyl moiety. The alkyl groups may contain optional non-carbon substituents like halogen substituents and optional functional groups like cyano-, carboxamide-, alkoxy-, protected hydroxy-groups or other functional groups known to those skilled in the art, and may also contain optional ether groups. The alkyl groups may also contain additional amino groups which may be primary, secondary or tertiary amino groups. The alkyl groups R, R′, and R″ may or may not be identical.

The term “alkyl” as used herein describes groups which are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The terms “carbocyclo” or “carbocyclic” as used herein alone or as part of another group denote optionally substituted, aromatic or non-aromatic, homocyclic ring or ring system in which all of the atoms in the ring are carbon, with preferably 5 or 6 carbon atoms in each ring. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thio.

The term “heteroatom” refers to atoms other than carbon and hydrogen.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms and/or 1 to 4 nitrogen atoms in the ring, and is bonded to the remainder of the molecule through a carbon. Exemplary groups include furyl, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyridazinyl, carbazolyl, purinyl, quinolinyl, isoquinolinyl, imidazopyridyl, and the like. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thio.

The terms “heterocyclo” or “heterocyclic” as used herein alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group preferably has 1 or 2 oxygen atoms and/or 1 to 4 nitrogen atoms in the ring, and is bonded to the remainder of the molecule through a carbon or heteroatom. Exemplary heterocyclo groups include heteroaromatics as described above. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thio.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thio.

Having described the invention in detail, it will be apparent that modifications and variations are possible without departing from the scope of the invention defined in the appended claims.

### EXAMPLES

### Example 1

### General Protocol

An empty column is packed with an ion exchange support and the excess liquid is drawn off. The column is washed with 1 ml of 2M sodium chloride in 10% acetonitrile and then with 0.1M sodium carbonate. The amino modified oligomer is dissolved in 1 mL of 0.1 M sodium carbonate and is loaded onto the column. The column is then washed with 1 mL 0.1M sodium carbonate, followed by 1 mL of water, followed by 1 mL of DMSO. A 1 mg/100 μL solution of the NHS ester comprising a dye as in DMSO is then added into the column. The NHS ester is allowed to sit in the column for 45 minutes, then the excess is blown out of the column. The column is washed with 1 mL of water, followed by 1 mL of a solution of 80% acetonitrile/10% acetic acid/10% water, 1 mL 0.1M sodium carbonate, and is washed again with 1 mL of water. The conjugated oligonucleotide is eluted with 1 mL of 2M sodium chloride in 10% acetonitrile.

### Example 2

### ROX Conjugation

PL-SAX was added drop wise to a column outlet until it was filled. The column was washed with 1 mL of 0.1 M sodium hydroxide in 50% methanol/50% water. The column was then washed with 1 mL of 2M sodium chloride/10% acetonitrile. The column was then washed with 0.1 M sodium carbonate. A C6MMT amino-modified oligomer having the following sequence: ACGGTGATGACGCGAGCTACACCA (SEQ ID NO: 1) with a {BHQ2} at the 3′ end was treated in water at 80° C. for 20 minutes, then was diluted with 0.1 M sodium carbonate, and passed through the column three times. The column was then washed with 1 mL of sodium carbonate and the wash was discarded.

The column was then washed with 1 mL DMSO. With the DMSO in the column 33 μL of a solution of ROX NHS ester in a concentration of 1 mg/100 μL was added to the column. The ROX NHS ester was allowed to stand in the column for 1 hour. The column was then washed with 1 mL of water. Following that, the column was washed with 1 mL of 80% acetonitrile in 10% acetic acid. The column was then washed with 1 mL of water, followed by 1 mL of 0.1M sodium carbonate. The conjugated ‘5-{ROX}, 3′-{BHQ2} oligonucleotide was eluted with 1 mL 2M sodium chloride and 10% acetonitrile and purified by HPLC.

### Example 3

### TAM Conjugation

PL-SAX was added drop wise to a column outlet until it was filled. The column was washed with 1 mL of 0.1 M sodium hydroxide in 50% methanol/50% water. The column was then washed with 1 mL of 2M sodium chloride/10% acetonitrile. The column was then washed with 0.1 M sodium carbonate. A C6MMT amino-modified oligomer having the following sequence: GGCAACACAAGTGAACTGC (SEQ ID. NO: 2) was treated in water at 80° C. for 20 minutes, then was diluted with 0.1 M sodium carbonate, and passed through the column three times. The column was then washed with 1 mL of sodium carbonate and the wash was discarded.

The column was then washed with 1 mL DMSO. With the DMSO in the column 33 μL of a solution of TAM NHS ester in a concentration of 1 mg/100 μL was added to the column. The TAM NHS ester was allowed to stand in the column for 1 hour. The column was then washed with 1 mL of water. Following that, the column was washed with 1 mL of 80% acetonitrile in 10% acetic acid. The column was then washed with 1 mL of water, followed by 1 mL of 0.1M sodium carbonate. The conjugated 5′-{TAM} oligonucleotide was eluted with 1 mL 2M sodium chloride and 10% acetonitrile and purified by HPLC.

### Example 4

### A488 Conjugation

PL-SAX was added drop wise to a column outlet until it was filled. The column was washed with 1 mL of 0.1 M sodium hydroxide in 50% methanol/50% water. The column was then washed with 1 mL of 2M sodium chloride/10% acetonitrile. The column was then washed with 0.1 M sodium carbonate. A C6MMT amino-modified oligomer having the following sequence: GCAGCCACCCGTAGGTGT (SEQ ID NO: 3) was treated in water at 80° C. for 20 minutes, then was diluted with 0.1 M sodium carbonate, and passed through the column three times. The column was then washed with 1 mL of sodium carbonate and the wash was discarded.

The column was then washed with 1 mL DMSO. With the DMSO in the column 33 μL of a solution of A488 NHS ester in a concentration of 1 mg/100 μL was added to the column. The A488 NHS ester was allowed to stand in the column for 1 hour. The column was then washed with 1 mL of water. Following that, the column was washed with 1 mL of 80% acetonitrile in 10% acetic acid. The column was then washed with 1 mL of water, followed by 1 mL of 0.1M sodium carbonate. The conjugated 5′-{A488} oligonucleotide was eluted with 1 mL 2M sodium chloride and 10% acetonitrile and purified by HPLC.

### Example 5

### Cy5 Conjugation

PL-SAX was added drop wise to a column outlet until it was filled. The column was washed with 1 mL of 0.1 M sodium hydroxide in 50% methanol/50% water. The column was then washed with 1 mL of 2M sodium chloride/10% acetonitrile. The column was then washed with 0.1 M sodium carbonate. A C6MMT amino-modified oligomer having the following sequence: AAAATTCCCCTCCACAATTA (SEQ ID NO: 4) was treated in water at 80° C. for 20 minutes, then was diluted with 0.1 M sodium carbonate and passed through the column three times. The column was then washed with 1 mL of sodium carbonate and the wash was discarded.

The column was then washed with 1 mL DMSO. With the DMSO in the column 33 μL of a solution of a Cy5 NHS ester in a concentration of 1 mg/100 μL was added to the column. The Cy5 NHS ester was allowed to stand in the column for 1 hour. The column was then washed with 1 mL of water. Following that, the column was washed with 1 mL of 80% acetonitrile in 10% acetic acid. The column was then washed with 1 mL of water, followed by 1 mL of 0.1M sodium carbonate. The conjugated ‘5-{Cy5} oligonucleotide was eluted with 1 mL 2M sodium chloride and 10% acetonitrile and purified by HPLC.

### Example 6

### DIG Conjugation

PL-SAX was added drop wise to a column outlet until it was filled. The column was washed with 1 mL of 0.1 M sodium hydroxide in 50% methanol/50% water. The column was then washed with 1 mL of 2M sodium chloride/10% acetonitrile. The column was then washed with 0.1 M sodium carbonate. A C6MMT amino-modified oligomer having the following sequence: AGCACTGCCACTGCTGCGGTTTCCCCAACA (SEQ ID NO: 5) was treated in water at 80° C. for 20 minutes, then was diluted with 0.1 M sodium carbonate and passed through the column three times. The column was then washed with 1 mL of sodium carbonate and the wash was discarded.

The column was then washed with 1 mL DMSO. With the DMSO in the column 33 μL of a solution of DIG NHS ester in a concentration of 1 mg/100 μL was added to the column. The DIG NHS ester was allowed to stand in the column for 1 hour. The column was then washed with 1 mL of water. Following that, the column was washed with 1 mL of 80% acetonitrile in 10% acetic acid. The column was then washed with 1 mL of water, followed by 1 mL of 0.1M sodium carbonate. The conjugated ‘5-{DIG} oligonucleotide was eluted with 1 mL 2M sodium chloride and 10% acetonitrile and purified by HPLC.

### Example 7

### ROX Conjugation

PL-SAX was added drop wise to a column outlet until it was filled. The column was washed with 1 mL of 0.1 M sodium hydroxide in 50% methanol/50% water. The column was then washed with 1 mL of 2M sodium chloride/10% acetonitrile. The column was then washed with 0.1 M sodium carbonate. A C6MMT amino-modified oligomer having the following sequence: GGCCTCACCCAGTCTACATT (SEQ ID NO: 6) was treated in water at 80° C. for 20 minutes, then was diluted with 0.1 M sodium carbonate and passed through the column three times. The column was then washed with 1 mL of sodium carbonate and the wash was discarded.

The column was then washed with 1 mL DMSO. With the DMSO in the column 33 μL of a solution of ROX NHS ester in a concentration of 1 mg/100 μL was added to the column. The ROX NHS ester was allowed to stand in the column for 1 hour. The column was then washed with 1 mL of water. Following that, the column was washed with 1 mL of 80% acetonitrile in 10% acetic acid. The column was then washed with 1 mL of water, followed by 1 mL of 0.1M sodium carbonate. The conjugated 5′-{ROX} oligonucleotide was eluted with 1 mL 2M sodium chloride and 10% acetonitrile and purified by HPLC.

### Example 8

### TAM Conjugation

PL-SAX was added drop wise to a column outlet until it was filled. The column was washed with 1 mL of 0.1 M sodium hydroxide in 50% methanol/50% water. The column was then washed with 1 mL of 2M sodium chloride/10% acetonitrile. The column was then washed with 0.1 M sodium carbonate. A C6MMT amino-modified oligomer having the following sequence: TGCTGATTGTTATCTAGTATGG (SEQ ID NO: 7) with a 3′-{BHQ2} was treated in water at 80° C. for 20 minutes, then was diluted with 0.1 M sodium carbonate and passed through the column three times. The column was then washed with 1 mL of sodium carbonate and the wash was discarded.

The column was then washed with 1 mL DMSO. With the DMSO in the column 33 μL of a solution of TAM NHS ester in a concentration of 1 mg/100 μL was added to the column. The TAM NHS ester was allowed to stand in the column for 1 hour. The column was then washed with 1 mL of water. Following that, the column was washed with 1 mL of 80% acetonitrile in 10% acetic acid. The column was then washed with 1 mL of water, followed by 1 mL of 0.1M sodium carbonate. The conjugated 5′-{TAM}, 3′-{BHQ2} oligonucleotide was eluted with 1 mL 2M sodium chloride and 10% acetonitrile and purified by HPLC.

### Example 9

### LC610 Conjugation

PL-SAX was added drop wise to a column outlet until it was filled. The column was washed with 1 mL of 0.1 M sodium hydroxide in 50% methanol/50% water. The column was then washed with 1 mL of 2M sodium chloride/10% acetonitrile. The column was then washed with 0.1 M sodium carbonate. A C6MMT amino-modified oligomer having the following sequence: CATCACCATCTTCATAGGCTACTTGACCTATAGT (SEQ ID NO: 8) with a 3′-{Phos} was treated in water at 80° C. for 20 minutes, then was diluted with 0.1 M sodium carbonate, and passed through the column three times. The column was then washed with 1 mL of sodium carbonate and the wash was discarded.

The column was then washed with 1 mL DMSO. With the DMSO in the column 50 μL of a solution of LC610 NHS ester diluted in 250 μL of DMSO was added to the column. The LC610 NHS ester was allowed to stand in the column for 1 hour. The column was then washed with 1 mL of water. Following that, the column was washed with 1 mL of 80% acetonitrile in 10% acetic acid. The column was then washed with 1 mL of water, followed by 1 mL of 0.1M sodium carbonate. The conjugated 5′-{LC610}, 3′-{Phos} oligonucleotide was eluted with 1 mL 2M sodium chloride and 10% acetonitrile and purified by HPLC.

### Example 10

### LC640 Conjugation

PL-SAX was added drop wise to a column outlet until it was filled. The column was washed with 1 mL of 0.1 M sodium hydroxide in 50% methanol/50% water. The column was then washed with 1 mL of 2M sodium chloride/10% acetonitrile. The column was then washed with 0.1 M sodium carbonate. A C6MMT amino-modified oligomer having the following sequence: AGGTGAACGGAAGTGCACACGGACC (SEQ ID NO: 9) with a 3′-{Phos} was treated in water at 80° C. for 20 minutes, then was diluted with 0.1M sodium carbonate, and passed through the column three times. The column was then washed with 1 mL of sodium carbonate and the wash was discarded.

The column was then washed with 1 mL DMSO. With the DMSO in the column 50 μL of a solution of LC640 NHS ester diluted in 250 μL of DMSO was added to the column. The LC640 NHS ester was allowed to stand in the column for 1 hour. The column was then washed with 1 mL of water. Following that, the column was washed with 1 mL of 80% acetonitrile in 10% acetic acid. The column was then washed with 1 mL of water, followed by 1 mL of 0.1M sodium carbonate. The conjugated 5′-{LC640} 3′ {Phos} oligonucleotide was eluted with 1 mL 2M sodium chloride and 10% acetonitrile and purified by HPLC.

### Example 11

### TxRd Conjugation

PL-SAX was added drop wise to a column outlet until it was filled. The column was washed with 1 mL of 0.1 M sodium hydroxide in 50% methanol/50% water. The column was then washed with 1 mL of 2M sodium chloride/10% acetonitrile. The column was then washed with 0.1M sodium carbonate. A C7 amino-modified oligomer having the following sequence: AAGCCGTCACGTAGTGCGCCA (SEQ ID NO: 10) with a 5′-{Btn} was diluted with 0.1 M sodium carbonate, and passed through the column three times. The column was then washed with 1 mL of sodium carbonate and the wash was discarded.

The column was then washed with 1 mL DMSO. With the DMSO in the column 33 μL of a solution of TxRd NHS ester in a concentration of 1 mg/100 μL was added to the column. The TxRd NHS ester was allowed to stand in the column for 1 hour. The column was then washed with 1 mL of water. Following that, the column was washed with 1 mL of 80% acetonitrile in 10% acetic acid. The column was then washed with 1 mL of water, followed by 1 mL of 0.1M sodium carbonate. The conjugated 5′-{Btn} 3′-{TxRd} oligonucleotide was eluted with 1 mL 2M sodium chloride and 10% acetonitrile and purified by HPLC.

What is claimed is:

1. A process for conjugating a N-hydroxysuccinimide ester with an amino-modified oligonucleotide, the process comprising contacting the N-hydroxysuccinimide ester with the amino-modified oligonucleotide, wherein the amino-modified oligonucleotide is non-covalently bound to an ion exchange support.

2. The process of claim 1, wherein the N-hydroxysuccinimide ester is of the Formula (I): 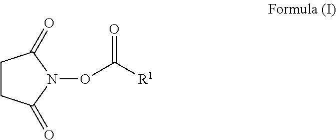
wherein R¹ is chosen from hydrocarbyl and substituted hydrocarbyl.

3. The process of claim 1, wherein R¹ comprises a fluorescent moiety, a dye, or a label.

4. The process of claim 1, wherein the amino-modified oligonucleotide comprises from about 5 to about 500 bases.

5. The process of claim 1, wherein the amino-modified oligonucleotide has a mass-average molecular weight of about 500 to about 100,000 Da.

6. The process of claim 1, wherein the amino-modified oligonucleotide has a mass-average molecular weight of about 1,500 to about 40,000 Da.

7. The process of claim 1, wherein the amino group comprising the amino-modified oligonucleotide is free during conjugation.

8. The process of claim 1, wherein conjugation is conducted in a non-aqueous environment.

9. The process of claim 1, wherein the ion exchange support comprises a strong base exchanger.

10. The process of claim 1, wherein the ion exchange support comprises a quaternary or tertiary amine.

11. A process for conjugating an N-hydroxysuccinimide ester with an amino-modified oligonucleotide, the process comprising contacting the N-hydroxysuccinimide ester with the amino-modified oligonucleotide bound to a solid support, wherein the process is conducted in a non-aqueous environment.

12. The process of claim 11, wherein the N-hydroxysuccinimide ester is of the Formula (I): 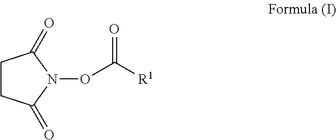
wherein R¹ is chosen from hydrocarbyl and substituted hydrocarbyl.

13. The process of claim 11, wherein R¹ comprises a fluorescent moiety, a dye, or a label.

14. The process of claim 11, wherein the amino group comprising the amino-modified oligonucleotide is free during conjugation.

15. A process for producing an oligonucleotide comprising an R¹ moiety, the process comprising:
(a) contacting an amino-modified oligonucleotide with an ion exchange support such that the amino-modified oligonucleotide binds to the support;
(b) contacting the supported amino-modified oligonucleotide with an N-hydroxysuccinimide ester comprising R¹; and
(c) eluting the oligonucleotide comprising R¹,
wherein R¹ is chosen from hydrocarbyl and substituted hydrocarbyl.

16. The process of claim 15, wherein R¹ comprises a fluorescent moiety, a dye, or a label.

17. The process of claim 15, wherein the process is conducted in an organic solvent.

18. The process of claim 15, wherein step (b) is conducted in a non-aqueous environment.

19. The process of claim 15, wherein the amino-modified oligonucleotide has a mass-average molecular weight of about 500 to about 100,000 Da.

20. The process of claim 15, wherein the amino-modified oligonucleotide has a mass-average molecular weight of about 1,500 to about 40,000 Da.
